(19) United States
(12) Patent Application Publication
(10) Pub. No.: US 2018/0085384 A1
(43) Pub. Date: Mar. 29, 2018

(54) NUTRITIONAL COMPOSITIONS AND INFANT FORMULAS CONTAINING OLIGOFRUCTOSE FOR REDUCING THE LOAD OF PATHOGENIC BACTERIA IN THE GUTS OF INFANTS AND YOUNG CHILDREN

(71) Applicant: NESTEC S.A., Vevey (CH)

(72) Inventors: Bernard Berger, Maracon (CH); David Frantz, Jeffersonville, PA (US); Susan Wernimont, Eagleville, PA (US); Manjiang Yao, Chester Springs, PA (US)

(21) Appl. No.: 15/573,665

(22) PCT Filed: Jun. 16, 2016
(86) PCT No.: PCT/EP2016/063930

§ 371 Date: Nov. 13, 2017

(30) Foreign Application Priority Data
Jun. 23, 2015 (EP) 15173474.6

(51) Int. Cl. A61K 31/702; A23L 29/30; A23L 33/19; A23L 33/00; A61K 9/00; A61K 31/23; A61K 38/38

(52) U.S. Cl. CPC A61K 31/702; A23L 29/30; A23L 33/19; A23L 33/30; A23V 2002/00; A61K 9/0095; A61K 31/23; A61K 38/38; A23L 33/40

(57) ABSTRACT

The invention relates to a nutrition composition with oligofructose and optionally high sn-2 palmitate for down regulating, decreasing or inhibiting the growth of pathogenic bacterial populations in the guts of infants or young children. The infants can be 0-12 months of age and the composition can be an infant formula. Higher dosage of oligofructose intensifies the effect. The composition promotes a healthy intestinal microbiota.

### FIELD OF THE INVENTION

The present invention relates to nutritional compositions for infants and young children and their health effects in infants. In particular, it relates to infant formula comprising a relatively high amount of oligofructose and optionally a relatively high amount of high sn-2 palmitate triglycerides.

### BACKGROUND TO THE INVENTION

Whenever mothers cannot breast-feed their infants, infant formula provides a suitable alternative to natural breast feeding with human breast milk. Nutritional compositions for infants and young children are often sold as powders to be reconstituted with water or in some instances as ready to drink or concentrated liquid compositions. Those compositions are intended to cover most or all the nutritional needs of the infants or young children.

It is known however, that human breast milk represents the ultimate gold standard in terms of infants' nutrition. Infant formula manufacturers have made many attempts to induce nutritional health effects close to or similar to the benefits of human breast milk.

Mother's milk is recommended for all infants. However, in some cases breast feeding is inadequate or unsuccessful for medical reasons or the mother chooses not to breast feed. Infant formulae have been developed for these situations. Fortifiers have also been developed to enrich mother's milk or infant formula with specific ingredients.

In many instances however, studies have shown that infant formula do not induce the identical effects on the body compared to human breast milk. For example, infants fed infant formula and infants fed human-breast milk (HBM) can exhibit a different intestinal microbiota.

A healthy intestinal flora is an indicator of the health of an infant and altered intestinal microbiota can be an indicator (and/or a cause) of abnormal health events such as diarrhoea, under-absorption of nutrients, colic, altered sleep, and altered growth and development.

It is known that the mode of delivery can affect the initial gut microbiota of infants: infants delivered by Caesarean section (C-section) have been shown to have a different gut microbiota compared to vaginally-delivered infants.

It is known that, among other ingredients, non-digestible carbohydrates (prebiotics) in particular can affect the promotion of particular microbiota. For example, it has been shown that certain galacto-oligosaccharides (GOS) and/or certain fructo-oligosaccharides (FOS) can promote the growth and prevalence of bifidobacteria in the gut, especially in infants.

The gut microbiota and its evolution during the development of the infant is, however, a fine balance between the presence and prevalence (amount) of many populations of gut bacteria. Some gut bacteria are classified as “generally positive” while other are “generally negative” (or pathogenic) as to their effect on the overall health of the infant.

Certain species of bacteria, such as bifidobacteria, may be under-represented in infants fed conventional infant formula in comparison to breast fed infants. Similarly some bacterial populations are considered pathogenic and should remain of low prevalence in the gut microbiota.

While many studies have identified ways to promote the growth and prevalence of positive bacteria in the gut of infants, little is known about ways to reduce the growth and prevalence of pathogenic bacteria in the gut of infants and young children.

There is a need to reduce or supress the growth of pathogenic bacteria in the guts of infants and young children.

There is a need to down-regulate the development and growth of pathogenic bacteria or “negative bacteria” in the gut of infants and young children.

There is a need to selectively affect the growth of pathogenic bacteria in the gut of infants and young children while not impacting the growth of positive gut bacteria.

There is a need to enhance a good balance in the overall gut microbiota of infants and young children. There is a need to induce such good balance such as to positively influence the overall health and development of the infants and young children.

There is a need to enhance a good balance in the overall gut microbiota of infants, especially by down-regulating or repressing the growth of pathogenic bacteria, during the first weeks of life when such a balance is being established.

There is a need to enhance such good balance by well tolerated, “soft-impact”, means having no or little side effects.

There is a need to negatively impact the growth of pathogenic bacteria in the gut of infants and young children, especially while avoiding the promotion of hard stools, cramps and colic.

WO2013068879A2, by Manjiang Yao et al., published on May 16, 2013, reports the effect of high sn-2 palmitate diets (in presence or absence of oligofructose) on the stools softness and on the induction of positive bacteria such as bifidobacteria in infants.

### SUMMARY OF THE INVENTION

The invention relates to a nutritional composition for infants and young children, such as an infant formula or follow-on formula. The composition can be in a powdered form or in a liquid form. When in a ready-to-drink liquid form, the composition comprises at least 3 g/L or at least 5 g/L of Oligofructose. When in powdered form or a concentrated liquid form, i.e that requires dilution before consumption, the composition comprises a sufficient amount of oligofructose to obtain respectively at least 3 g/L or 5 g/L of oligofructose in the reconstituted composition. Preferably the composition is in a powdered form.

The composition of the invention has the effect of down-regulating, decreasing or inhibiting the growth as well as reducing the abundance of pathogenic bacteria in the gut of infants or young children. Preferably the infants or young children are infants 0-12 months of age.

### BRIEF DESCRIPTION OF THE FIGURES

FIGS. 1 and 2 illustrate the effect of various nutritional compositions on the microbiota of infants.

FIG. 3 illustrates the change from baseline in selected faecal pathogenic bacteria species concentrations, by feeding group. The represented bacterial populations comprises both non-pathogenic, potentially pathogenic and pathogenic bacterial populations, as well as the total bacteria.

FIG. 4 illustrates the change from baseline in total measured pathogens, by feeding group. The change in the adjusted mean faecal counts of total measured pathogens [summed counts of all 4 pathogens including Escherichia coli (EPEC), Klebsiella pneumoniae, Clostridium difficile and Clostridium perfringens] was calculated as the difference between the log at baseline and week 8 for the total pathogen counts.

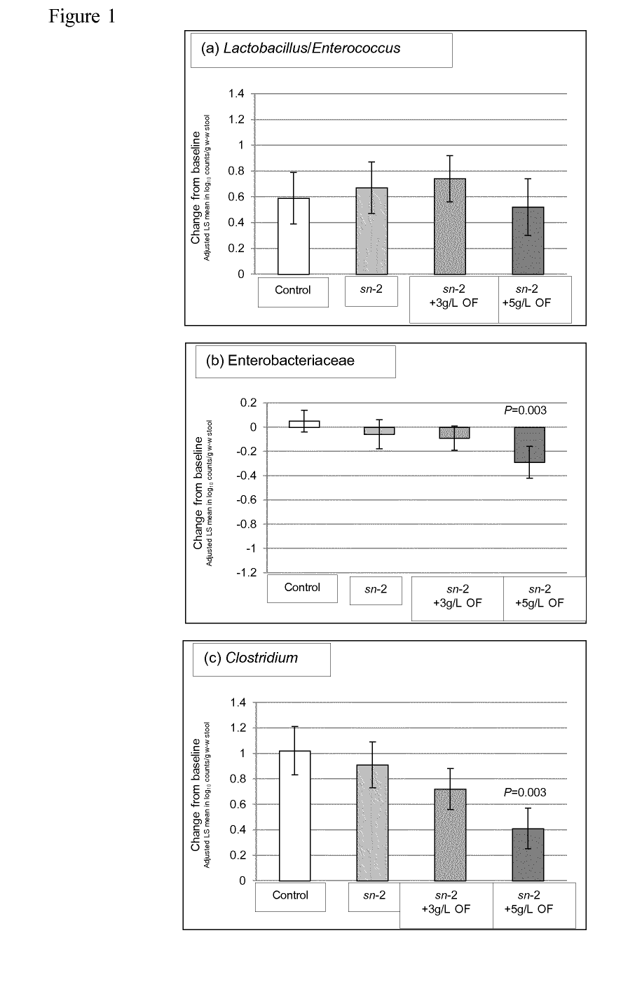

In all 4 figures (FIG. 1,2,3,4) data illustrate the change from baseline in faecal bacteria group concentrations, by feeding group. Data shown represent the difference (baseline to week 8) in the adjusted LS mean log₁₀ transformed bacteria concentrations (expressed as counts/g stool wet weight). P value represents comparison to Control.

### DESCRIPTION OF THE INVENTION

### Definitions

As used herein, the following terms have the following meanings.

The term “infant” means a child under the age of 12 months.

The expression “young child” means a child aged between one and three years, also called toddler.

An “infant or young child born by C-section” means an infant which was delivered by caesarean section. It means that the infant was not vaginally delivered.

A “preterm” or “premature” means an infant or young child that was not born at term. Generally it refers to an infant born prior to 36 weeks of gestation.

The expression “nutritional composition” means a composition which nourishes a subject. This nutritional composition is usually to be taken enterally, orally, parenterally or intravenously, and it usually includes a lipid or fat source and a protein source. Preferably, a nutritional composition is for oral use.

The expression “hypoallergenic nutritional composition” means a nutritional composition which is unlikely to cause allergic reactions.

The expression “synthetic composition” means a mixture obtained by chemical and/or biological means, which can be chemically identical to the mixture naturally occurring in mammalian milks.

The expression “infant formula” means a foodstuff intended for particular nutritional use by infants during the first four to six months of life and satisfying by itself the nutritional requirements of this category of person (Article 1.2 of the European Commission Directive 91/321/EEC of May 14, 1991 on infant formulae and follow-on formulae).

The expression “starter infant formula” means a foodstuff intended for particular nutritional use by infants during the first four months of life.

The expression “follow-on formula” means a foodstuff intended for particular nutritional use by infants aged over four months and constituting the principal liquid element in the progressively diversified diet of this category of person.

The expression “baby food” means a foodstuff intended for particular nutritional use by infants during the first years of life.

The expression “fortifier” refers to liquid or solid nutritional compositions suitable for mixing with breast milk or infant formula.

The term “weaning period” means the period during which the mother's milk is substituted by other food in the diet of an infant.

The “mother's milk” should be understood as the breast milk or colostrum of the mother (=Human Breast Milk=HBM).

The term “oligofructose” as used herein refers to a fructose oligomers. It can be long chain or short chain, pending on the degree of polymerization of the oligofructose (number of monomers). Preferably the oligofructose of the invention is a short-chain oligofructose, most preferably it has a degree of polymerization of from 2 to 10, for example a degree of polymerization of from 2 to 8.

The term “sn-2 palmitate” as used herein refers to palmitic acid in the sn-2 position of the triglyceride to which it is bonded.

“High sn-2 palmitate triglyceride” refers to a triglyceride (TG) containing more than 30% of the palmitic acids in the sn-2 position. For example a commercially available high sn-2 palmitate ingredient is sold by Lipid Nutrition is Betapol™ B-55. It is a triglyceride mixture derived from vegetable oil in which at least 54% of the palmitic acid is in the sn-2 position of the glycerol molecule.

“Alpha-Lactalbumin” refers to a high-quality, easy-to-digest whey protein that comprises 20-25% of total human breast milk (HBM) protein and is the primary protein found in HBM. The structure of alpha-lactalbumin is comprised of 123 amino acids and 4 disulfide bridges and the protein has a molecular weight of 14.2K Daltons. Alpha-lactalbumin is ideal for lower protein infant formulas due to its high content of essential amino acids, particularly tryptophan.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

The term “cfu” should be understood as colony-forming unit.

All percentages are by weight unless otherwise stated.

The invention will now be described in further details. It is noted that the various aspects, features, examples and embodiments described in the present application may be compatible and/or combined together.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

### DETAILED DESCRIPTION OF THE INVENTION

Typically, an infant formula in a ready-to-consume liquid form (for example reconstituted from a powder) provides 60-70 kcal/100 ml. Infant formula typically comprises, per 100 Kcal: about 1.8-4.5 g protein; about 3.3-6.0 g fat (lipids); about 300-1200 mg linoleic acid; about 9-14 g carbohydrates selected from the group consisting of lactose, sucrose, glucose, glucose syrup, starch, maltodextrins and maltose, and combinations thereof; and essential vitamins and minerals. Lactose may be the pre-dominant carbohydrate in an infant formula. For example, a liquid infant formula may contain about 67 kcal/100 ml. In some embodiments, infant formula may comprise about 1.8-3.3 g protein per 100 Kcal. Infant formula may be in the form of a powder which can be reconstituted into a ready-to-feed liquid by adding an amount of water that results in for example a liquid having about 67 kcal/100 ml.

An infant formula may also comprise nucleotides selected from cytidine 5′-monophosphate (CMP), uridine 5′-monophosphate (UMP), adenosine 5′-monophosphate (AMP), guanosine 5′-monophosphate (GMP) and inosine 5′-monophosphate (IMP), and mixtures thereof. Infant formula may also comprise lutein, zeaxanthin, fructo-oligosaccharides, galacto-oligosaccharides, sialyl-lactose, and/or fucosyl-lactose. Long chain polyunsaturated fatty acids, such as docosahexaenoic acid (DHA) and arachidonic acid (AA) may be included in infant formula. Infant formula may also include free amino acids. Infant formula may also include other ingredients well-known in the art.

In one embodiment, the infant formula of this invention comprises about 5-6 g per 100 kcal of fat (triglycerides), with at least about 7.5 wt % of this fat, for example about 7.5-12.0%, consisting of palmitic acid in the sn-2 position of a triglyceride. In some embodiments, about 7.8-11.8%, about 8.0-11.5 wt %, about 8.5-11.0% or about 9.0-10.0 wt % of the fat is palmitic acid in the sn-2 position of a triglyceride.

In some embodiments, palmitic acid comprises from about 15 to about 25%, such as from about 15 to about 20%, of the total fatty acids content of the formula, by weight, and at least from about 30%, for example, from about 35 to about 43% of the total palmitic acid content is in the sn-2 position.

In some embodiments, the infant formula further comprises at least one omega 6 fatty acid and at least one omega 3 fatty acid in a ratio of about 6 to about 1. In one embodiment, at least one omega 6 fatty acid comprises from about 10 to about 15% by weight of the total fatty acids and at least one omega 3 fatty acid comprises from about 1.2% to about 3.6% of the total fatty acids. In some embodiments, the infant formula comprises at least one omega 6 fatty acid present from about 2 to about 4% of the total weight and at least one omega 3 fatty acid present from about 0.3% to about 0.6% of the total weight.

The fat in the infant formula of this invention comprises a variety of triglycerides typically found in milk and/or infant formula. The most common fatty acid residues in the triglycerides are palmitic and oleic acids. Fatty acid residues in addition to oleic and palmitic acids that are present include, but are not limited to linoleic acid, alpha linolenic acid, lauric acid, myristic acid, docosahexaenoic acid, and arachidonic acid.

Recent infant clinical studies have shown that nutritional formulas containing at least one omega 6 fatty acid and at least one omega 3 fatty acid in a ratio of from about 6 to about 1 increased DHA accretion in erythrocytes and plasma. A balanced ratio of about 6:1 of omega 6 fatty acid to omega 3 fatty acid may also provide long term health benefits including protection against cardiovascular disease. Such balance will be achieved by formulating the present invention with vegetable oil fat sources that have omega 6 fatty acid content, such as, for example, soybean oil and sunflower oil, and omega 3 fatty acid content, for example, rapeseed, canola, flaxseed, chia, perlla or walnuts. A unique fat blend with 5 different oils will be used to achieve the modified fat blend

In one embodiment, the infant formula of this invention comprises from about 1.8 to about 2.2 g of total protein per 100 kcal, for example, about from 1.8 to about 2.1 g or from about 1.9 to about 2.1 g protein per 100 kcal, wherein from about 0.3 to about 0.4 g/100 kcal of protein is alpha-lactalbumin. The infant formula of this invention may be in the form of a ready-to-feed liquid, or may be a liquid concentrate or powdered formula that can be reconstituted into a ready-to-feed liquid by adding an amount of water that results in a liquid having about 67 kcal/100 ml. The infant formula of this invention includes all the ingredients that are required by law in the US or EU, including but not limited to certain vitamins, minerals, and essential amino acids. It may also include nucleotides, such as CMP, UMP, AMP, GMP and IMP, lutein, zeaxanthin, and other ingredients known in the art.

### Oligofructose (OF)

The infant formula of this invention can comprise at least about 0.4 g or at least 0.7 g of oligofructose per 100 kcal of the composition. In some embodiments, it contains from about 0.4 to about 0.9 g, from about 0.4 to about 0.7 g, from about 0.4 to about 0.5 g, from about 0.7 to about 0.8 g, or from about 0.7 to about 0.9 g, oligofructose per 100 kcal.

In some embodiments the oligofructose has a degree of polymerization of from 2 to 10. In some embodiments, at least 80%, 90%, 95%, 99% or 100% of the oligofructose has a degree of polymerization of from 2 to 8 (between 2 and 8).

In one embodiment the composition of the invention comprises
at least 3 g/L or at least 5 g/L of Oligofructose (OF) when the composition is a ready-to-drink liquid composition, or
a sufficient amount of oligosaccharide to obtain respectively at least 3 g/L or 5 g/L of oligofructose in the reconstituted composition when said nutritional composition is a powered or concentrated composition.

In some embodiments the nutritional composition of the invention comprises at least 0.4 g OF/100 kcal of composition or at least 0.7 g, or at least 0.75 g, or at least 0.8 g or at least 0.9 g OF/100 kcal of composition.

It is generally admitted, in view of the results illustrated by the examples, that a high amount of oligofructose delivers a stronger effect. A upper limit for a beneficial effect of oligofructose may; however, exist when disadvantageous side effect begins. Such upper limit may be for example 2.2 g/100 kcal, 2.0 g/100 kcal, 1.8 g/100 kcal, 1.5 g/100 kcal, or 1.2 g/100 kcal. Preferably the composition of the invention comprises 5 g OF/L or 0.75 or 0.9 g OF/100 kcal of composition or at last such amounts.

### Health Effect

The composition of the invention has a positive effect on the microbiota of the subject infants or young children. Such positive effect is characterized by the down regulation, decrease or inhibition of growth of pathogenic bacteria. Such pathogenic bacteria can be naturally localized in the gut or can have an exogenous origin. Such down regulation, decrease or inhibition of growth can be measured, for example, by the analysis of the stools. Such down regulation, decrease or inhibition of growth can be measured preferably at 4 weeks, 8 weeks, 12 weeks 16 weeks or 24 weeks of age. Such down regulation, decrease or inhibition of growth can, for example, be evaluated by comparison to the gut microbiota of infants fed a conventional nutritional composition (e.g. infant formula) not comprising the oligofructose present in the invention.

The composition of the invention has an overall effect on a vast variety of pathogenic gut bacteria. In various embodiments of the invention the down regulation, decrease or inhibition of growth is particularly effective (visible/measurable) on Klebsiella pneumoniae and/or E. coli, and/or Enteropathogenic E. coli, and/or Clostridium difficile and/or Clostridium perfringens, as well as total measured pathogens (see Figures).

By down-regulating, decreasing and/or inhibiting the growth of populations of pathogenic bacteria, the composition of the invention provides positive health effects and contributes to the induction and maintenance of a healthy intestinal microbiota. Such a healthy gut/intestinal microbiota is ultimately linked to proper nutrient absorption, adequate growth, less colic, less diarrhoea and the best gut health.

The effect of the invention can be preventive (for example avoiding the imbalance of the gut microbiota, avoiding gut infections, maintaining a healthy intestinal microbiota, inducing a healthy intestinal microbiota) or curative (restoring a healthy gut microbiota when it is impaired, helping eliminate or decrease pathogenic populations in the gut/intestine, inducing a healthy microbiota after impairments due, for example, to diarrhoea or infections.

In one embodiment the composition of the invention is characterized in that the health effect observed on the microbiota (ie.e down-regulation, decrease or inhibition the growth of pathogenic bacteria in the gut of infants or young children) further induces the gut microbiota to be similar or more similar to the microbiota of exclusively breast-fed infants or young children (when compared to the microbiota of infants fed from a conventional composition—i.e. a composition not containing the essential features of the invention, for example not containing oligofructose). It is understood that inducing a gut microbiota which resemble/is similar to the gut microbiota of breast fed infants is beneficial as the human breast milk represents the gold standard of nutrition for infants.

### Target Infants

In one embodiment, the infants or young children are born at term. All infants can benefit from the invention as all infants are or can be, at a certain age, susceptible to acquiring an unbalanced intestinal/gut microbiota. In one embodiment, the infants or young children are born pre-mature (preterm). In one embodiment, the infants or young children are vaginally delivered. In one embodiment, the infants or young children are delivered by C-section. It is foreseen that the composition of the invention may be even more beneficial to infants born with possibly impaired gut microbiota or fragile infants (such as prematurely born infants and/or infants born by C-section). It is also foreseen that the composition of the invention may be even more beneficial to infants exhibiting intestinal disorders (such as diarrhea, infections or colic) after birth, for example, during the first 4 weeks after birth.

In embodiments of the invention, the infants are born prematurely or born by caesarean section, or exhibit unbalanced or abnormal intestinal microbiota or suffer from intestinal infection; optionally, said above conditions are targeted by the composition of the invention when the infants are 0-6 months of age. Without being bound by the theory, it is believed that younger infants benefit even more from the invention, especially when the infants have (or are at risk of having) an “unbalanced intestinal microbiota” and/or have a fragile health condition (as exemplified by the conditions cited above).

In one embodiment, the infants and young children are 0-6-months, or 0-12 months or 0-36 months of age. It is foreseen that the composition of the invention may be even more beneficial to infants just after birth (0-4 weeks or 0-8 weeks) as their intestinal tract may be more fragile.

The following examples are presented to illustrate certain embodiments and features of the present invention, but should not be construed as limiting the scope of this invention.

### Intended Feeding Regimen:

In one embodiment, the composition of the invention is fed to the infant or young children (or intended to be fed or instructed to be fed) during 2, 4, 8, 12 weeks or during at least 2, 4, 8, 12 weeks. In preferable embodiments, it is fed (or intended to be fed or instructed to be fed) during the first 4, 8 or 12 weeks of the life of the infant. It is believed that starting early (at birth or close to birth) is preferred to induce the intended effect.

### Proteins/Alpha-Lactalbumin

The composition of the invention comprises a source of protein. Such protein source can, for example, deliver between 1.6.c and 3 g protein/100 kcal. In one embodiment intended for premature infants, such amount can be between 2.4 and 4 g/100 kcal or more than 3.6 g/100 kcal. In one embodiment, the amount can be below 2.0 g per 100 kcal, e.g. in an amount below 1.8 g per 100 kcal.

The type of protein is not believed to be of highest criticality to the present invention provided that the minimum requirements for essential amino acid content are met and satisfactory growth is ensured. However particular proteins can provide a most suitable substrate for the microbiota. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

Preferably the protein source is whey predominant (more than 50% of proteins are coming from whey proteins). In one embodiment, the protein of the composition are intact proteins or mostly (more than 90%) intact proteins.

The proteins may be intact or hydrolysed or a mixture of intact and hydrolysed proteins. By the term “intact” is meant that the main part of the proteins are intact, i.e. the molecular structure is not altered, for example, at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. It may be desirable to supply partially hydrolysed proteins (degree of hydrolysis between 2 and 20%), for example, for infants believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In one preferable embodiment, the proteins of the composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 90. It is understood that hydrolysed proteins can have several effects on allergy: hydrolyzed proteins can be less allergenic, hence triggering less immune allergic reactions. Hydrolyzed proteins, especially small peptides (of less than 20, 10 or 5 amino acids), can induce oral tolerance hence influencing the future allergic status of the subject. It is understood that hydrolyzed proteins can advantageously combine with the fucosylated oligosaccharide(s) of the present invention by providing a dual effect, possibly synergistic effect by acting at least at 2 different levels in the establishment of allergic symptoms or allergic status.

In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one embodiment, the hydrolyzed proteins are the sole source of protein (i.e. 100% or at least 90% of protein are hydrolyzed).

In one embodiment, the hydrolyzed proteins are the primary source of protein (i.e. at least 50%, preferably 60% of proteins are hydrolyzed).

In one embodiment, the nutritional composition of the invention comprise alpha-lactalbumin in an amount of at least 0.2 or 0.3 or 0.4 g/100 kcal or at least 1.7 g, or 2.0 or 2.3, or 2.6 g/L. The presence of alpha-lactalbumin in a certain amount is believed to enhance the effect of the oligofructose by providing, for example, an adequate nutritional substrate to the microbiota.

### Further Prebiotics:

The composition of the invention can also comprise further non-digestible oligosaccharides (e.g. prebiotics). They are usually in an amount between 0.3 and 10% by weight of composition.

Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such further fructo-oligosaccharides (FOS) and/or galacto-oligosaccharides (GOS). A combination of prebiotics may be used such as 90% GOS with 10% short chain fructo-oligosaccharides. Another combination of prebiotics is 70% short chain fructo-oligosaccharides and 30% inulin (=long chain FOS). Both, as well as oligofructose (OF), are available commercially, in particular from the company BENEO (Beneo GmbH, Maximilianstrasse, 68165, Mannheim, Germany).

### Preferred Nutritional Composition Matrix:

The composition according to the invention can be a synthetic nutritional composition. It can be an infant formula, a starter infant formula, a follow-on formula or a fortifier such as a human milk fortifier, or a supplement. Preferably the composition of the invention is an infant formula, or a fortifier or a supplement intended for the first 4 or 6 months of age.

Fat/High sn-2 Palmitate:

In one embodiment, the nutritional composition comprises triglycerides with high sn-2 palmitate, preferably triglycerides having more than 33% of the palmitic acids in sn-2 position.

In one embodiment, the infant formula of this invention comprises about 5-6 g per 100 kcal of fat (triglycerides), with at least about 7.5 wt % of this fat, for example, about 7.5-12.0%, consisting of palmitic acid in the sn-2 position of a triglyceride. In one embodiment, of the invention the composition comprises at least 7.5%, preferably 8%, more preferably at least 9.6% of the fat is sn-2 palmitate.

In some embodiments, about 7.8-11.8%, about 8.0-11.5 wt %, about 8.5-11.0% or about 9.0-10.0 wt % of the fat is palmitic acid in the sn-2 position of a triglyceride.

In some embodiments, palmitic acid comprises from about 15 to about 25%, such as from about 15 to about 20%, of the total fatty acids content of the formula, by weight, and at least from about 30%, for example, from about 35 to about 43% of the total palmitic acid content is in the sn-2 position.

In some embodiments, the infant formula further comprises at least one omega 6 fatty acid and at least one omega 3 fatty acid in a ratio of about 6 to about 1. In one embodiment, at least one omega 6 fatty acid comprises from about 10 to about 15% by weight of the total fatty acids and at least one omega 3 fatty acid comprises from about 1.2% to about 3.6% of the total fatty acids. In some embodiments, the infant formula comprises at least one omega 6 fatty acid present from about 2 to about 4% of the total weight and at least one omega 3 fatty acid present from about 0.3% to about 0.6% of the total weight.

The fat in the infant formula of this invention comprises a variety of triglycerides typically found in milk and/or infant formula. The most common fatty acid residues in the triglycerides are palmitic and oleic acids. Fatty acid residues in addition to oleic and palmitic acids that are present include, but are not limited to linoleic acid, alpha linolenic acid, lauric acid, myristic acid, docosahexaenoic acid, and arachidonic acid.

A commercially available composition sold by Lipid Nutrition is Betapol™ B-55, which is a triglyceride mixture derived from vegetable oil in which at least 54% of the palmitic acid is in the sn-2 position of the glycerol molecule. In one embodiment, the fat content of the composition of the invention is about 40-50% Betapol™ B-55 by weight, for example, from about 43% to about 45% by weight. Those skilled in the art will appreciate that the percentage of the high sn-2 fat used and the total amount of sn-2 palmitate in the formula may vary, and that a different high sn-2 palmitate oil may be used, without departing from the spirit and scope of the invention.

Although feeding an infant a formula containing a high percentage of sn-2 palmitate helps to produce softer stools and growth of bifidobacteria in the colon, the combination of high sn-2 palmitate with oligofructose provides significantly superior stool softening while inducing an optimal gut microbiota balance and enhanced down-regulation of pathogenic bacteria in the colon of formula-fed infants.

### Example 1

In the following experimental results the infant formula used were as follows. Compositions 3 and 4 are examples of the invention.

1. Control Formula (not a composition of the invention), coded “control formula”.

As a ready-to-feed liquid infant formula, the Control Formula has 670 kcal/L. Ingredients are shown below:

| Ingredients | Per 100 Kcal | Per Liter |
|---|---|---|
| Total Protein | 2.0 g | 13.4 g |
| (alpha-lactalbumin) | (0.3 g) | (2.3 g) |
| Total Fat | 5.4 g | 36 g |
| (sn-2 palmitate) | (0.19 g) | (1.3 g) |
| Total Carbohydrates | 11 g | 73 g |

The Control Formula also includes essential amino acids, minerals and trace elements, nucleotides, and various optional ingredients and food additives commonly used in infant formula.

2. High sn-2 Formula (not a composition of the invention), coded “sn-2 formula”.

This formula is the same as the Control Formula, except that 9.6 wt % of the fat is sn-2 palmitate. This is accomplished by using fat that is 57% vegetable oil and 43% Betapol™ B-55 in which about 55% of the palmitic acid is in the sn-2 position. (Betapol is commercially available from Loders Croklaan, Hogeweg 1, 1521 AZ Wormerveer, P.O. Box 4, 1520 AA Wormerveer, The Netherlands)

3. High sn-2+Oligofructose Formula A (composition of the invention) coded “sn-2+3 g/L OF formula”.

This formula is the same as the High sn-2 Formula except that it includes 3.0 g/L (0.4 g per 100 kcal) oligofructose.

(of note, another example of a composition of the invention is the same formula without the Betapol ingredient (which brings 9.6 wt % of the fat as sn-2 palmitate)
4. High sn-2+Oligofructose Formula B (composition of the invention), coded “sn-2+5 g/L OF formula”.

This formula is the same as the High sn-2 Formula except that it includes 5.0 g/L (0.7 g per 100 kcal) of oligofructose.

(of note, another example of a composition of the invention is the same formula without the Betapol ingredient (which brings 9.6 wt % of the fat as sn-2 palmitate)

A detailed listing of the ingredients of the compositions (1) “control formula”, (2) “sn-2” formula, (3) “sn-2+3 g/L OF formula” and (4) “sn-2+5 g/L OF formula”, is given in Table 1 below.

TABLE 1
Summary of the compositions

| | | Control | “sn2” | sn2 + 3 g/L | sn2 + 5 g/L |
|---|---|---|---|---|---|
| Per Liter | Units | formula | formula | OF formula | OF formula |
| Energy | Kcal | 670 | 670 | 670 | 670 |
| Protein | g | 13.4 | 13.4 | 13.4 | 13.4 |
| Fat | g | 36 | 36 | 36 | 36 |
| % C16 at sn-2 | % total | 2.6 | 9.6 | 9.6 | 9.6 |
| | fat | | | | |
| Carbohydrate | g | 73 | 73 | 73 | 73 |
| Oligofructose | g | 0 | 0 | 3 | 5 |
| Vitamin A | mcg | 660 | 660 | 660 | 660 |
| (RE) | | | | | |
| Vitamin D | mcg | 10.6 | 10.6 | 10.6 | 10.6 |
| Vitamin E | mg | 7.4 | 7.4 | 7.4 | 7.4 |
| (TE) | | | | | |
| Vitamin K | mcg | 67 | 67 | 67 | 67 |
| Vitamin B₁ | mcg | 1000 | 1000 | 1000 | 1000 |
| Vitamin B₂ | mcg | 1100 | 1100 | 1100 | 1100 |
| Vitamin B₆ | mcg | 550 | 550 | 550 | 550 |
| Vitamin B₁₂ | mcg | 1.8 | 1.8 | 1.8 | 1.8 |
| Niacin | mcg | 5000 | 5000 | 5000 | 5000 |
| Folic Acid | mcg | 107 | 107 | 107 | 107 |
| Pantothenic | mcg | 3500 | 3500 | 3500 | 3500 |
| Acid | | | | | |
| Biotin | mcg | 20 | 20 | 20 | 20 |
| Vitamin C | mg | 90 | 90 | 90 | 90 |
| Choline | mg | 100 | 100 | 100 | 100 |
| Inositol | mg | 45 | 45 | 45 | 45 |
| Taurine | mg | 47 | 47 | 47 | 47 |
| Lutein | mcg | 25 | 25 | 25 | 25 |
| Carotenes | mcg | 210 | 210 | 210 | 210 |
| Calcium | mg | 420 | 420 | 420 | 420 |
| Phosphorous | mg | 240 | 240 | 240 | 240 |
| Magnesium | mg | 45 | 45 | 45 | 45 |
| Iron | mg | 8 | 8 | 8 | 8 |
| Zinc | mg | 6 | 6 | 6 | 6 |
| Manganese | mcg | 50 | 50 | 50 | 50 |
| Copper | mcg | 333 | 333 | 333 | 333 |
| Iodine | mcg | 100 | 100 | 100 | 100 |
| Sodium | mg | 160 | 160 | 160 | 160 |
| Potassium | mg | 650 | 650 | 650 | 650 |
| Chloride | mg | 433 | 433 | 433 | 433 |
| Selenium | mcg | 14 | 14 | 14 | 14 |
| Fluoride | mcg | 25 | 25 | 25 | 25 |
| Nucleotides | mg | 26 | 26 | 26 | 26 |
| CMP | mg | 13 | 13 | 13 | 13 |
| UMP | mg | 5.0 | 5.0 | 5.0 | 5.0 |
| AMP | mg | 4.0 | 4.0 | 4.0 | 4.0 |
| GMP | mg | 2.0 | 2.0 | 2.0 | 2.0 |
| IMP | mg | 2.0 | 2.0 | 2.0 | 2.0 |

### Example 2—Clinical Study

### Design and General Overview

This study was an eight-week, randomized, controlled, double-blind clinical trial of formula-fed infants (n 300) and a non-randomized HM-fed reference group (n 75); The study included 3 clinic visits (baseline, week 4, and week 8) and 3 telephone calls (week 2, week 6, and a post-study follow-up call 2 weeks after the last clinic visit). At the baseline visit, participant demographics and household characteristics were collected. The present analysis focuses on a subset of infants (n 183), comprising 32-40 infants per group who provided stool samples at baseline (study day 1) and week 8 to be analysed for faecal microbiota composition and parameters of microbial metabolic activity (including pH and organic acids).

The study was conducted at Mutinlupa Health Centre, Mutinlupa City, in the Philippines between April and September 2009. This study was conducted according to the guidelines laid down in the Declaration of Helsinki and all procedures involving human subjects/patients were approved by the National Ethics Committee and the Bureau of Food and Drug in the Philippines. Written informed consent was obtained from the parent or legal guardian of each infant.

### Participants

Healthy, term (37-42 weeks gestation) singleton infants aged 7-14 days with weight-for-age ≧5th percentile according to Filipino reference standards were enrolled. Infants were required to be exclusively consuming and tolerating a cow's milk-based infant formula to be eligible for the formula-fed groups, or exclusively consuming HM to be eligible for the HM-fed group. Mothers were encouraged to breastfeed and were approached for participation in the formula-fed study arms only after the mother had made the decision to exclusively formula feed.

Infants were excluded from the study if they were presently receiving or had received medications which could potentially impact study endpoints; these included any medication known or suspected to affect fat digestion, absorption, and/or metabolism (e.g., pancreatic enzymes); any vitamin and/or mineral supplements which contain calcium; suppositories, bismuth-containing medications, herbal supplements, or medications that may neutralize or suppress gastric acid secretion. In addition, infants were excluded from the study if they were presently receiving or had received any antibiotics or antifungal medications (except topical) and HM-fed infants were excluded if their mothers were presently receiving or had received these medications post-partum.

Recruitment ended when the sample size goal was reached. The trial ended when the final subject completed all protocol requirements or withdrew from the study.

### Study Feedings

Formula-fed infants were randomized to receive ad libitum 1 of 4 formulas (i.e. compositions similar to the compositions of example 1): 1. a bovine milk-based, whey-predominant, alpha-lactalbumin-enriched term infant formula with a 100% vegetable fat blend (S-26 GOLD, Wyeth Nutrition, Askeaton, Ireland) (“Control”); 2. a high sn-2 palmitate formula (Control formula modified to contain 60% vegetable fat blend and 40% high sn-2 palmitate fat blend [Betapol™, Loders Croklaan, Wormerveer, the Netherlands]) (“sn-2”); 3. a high sn-2 palmitate formula supplemented with OF (Orafti® P95, BENEO-ORAFTI, Tienen, Belgium) at 3.0 g/L (“sn-2+3 g/L OF”); 4. a high sn-2 palmitate formula supplemented with OF (Orafti® P95) at 5.0 g/L (“sn-2+5 g/L OF”). Study formulas were produced in a powdered form and, except for the individual package number, were packaged in an identical manner. Instructions for formula preparation and storage were provided on the formula labels in both English and Filipino. All study formulas met Codex Alimentarius nutritional requirements for infant formula. Nutrient composition of the formulas has been previously described. Randomization was conducted using validated randomization software and infants were allocated to each of the 4 formula arms in approximately equal numbers, as follows: 75 infants to the Control formula, 74 infants to the sn-2 formula, 76 infants to the sn-2+3 g/L OF formula, and 75 infants to the sn-2+5 g/L OF formula. HM-fed infants (n 75) fed ad libitum were included as a nonrandomized reference group.

### Faecal Microbiota and Biochemistry

Stool samples were collected from a subset of infants at the baseline (study day 1) and week 8 clinic visits. Parents or legal guardians gave consent for the collection of these samples. At least 3 g of freshly passed stool was collected at the clinic by study personnel, using a scoop attached to the inner aspect of the lid of a sterile polypropylene vial, and immediately stored in a −20° C. freezer. Frozen samples were shipped on dry ice to NIZO Food Research B.V., Ede, The Netherlands.

Fluorescent in situ hybridization (FISH) analysis was used to determine concentrations of bacterial groups. Samples were fixed overnight in paraformaldehyde as described previously and stored prior to being shipped on dry ice to the Department of Medical Microbiology, University Medical Centre Groningen, Groningen, The Netherlands where FISH analyses were conducted For hybridizations with the Lab158 probe the samples were pre-treated with lysozyme and lipase to permeabilize the Gram-positive cell wall, as described elsewhere. Quantitative determination was conducted by an automated process; a cumulative CV was used to assess the quality of automated counting. If the cumulative CV was higher than 15%, the sample was recounted. Faecal bacterial group data are reported as log₁₀ transformed counts/g stool wet weight.

To further investigate changes seen in faecal bacterial groups, an exploratory analysis of specific faecal bacterial pathogens was performed. Real-time PCR detection was performed using commercially available kits. Targets included: Klebsiella pneumoniae, Clostridium difficile (toxin B), and Clostridium perfringens (all strains) (PrimerDesign, Southampton, United Kingdom) and Enteropathogenic Escherichia coli (EPEC) (Shanghai Z J Biotech Co., Shanghai, China). Faecal bacterial pathogen data are expressed as log₁₀ transformed gene copy number counts/g stool wet weight.

Faecal organic acids were determined by HPLC as previously described and are presented as μmol/g stool wet weight. The lower limit of detection of each faecal organic acid varied depending on the background reading for that acid in the sample matrix, ranging from 40 μg/g to 400 μg/g stool wet weight. The CV for all of the acids analysed ranged from 3.2% to 5.0%. Quality control was achieved by including two standard samples containing different concentrations of the main organic acids in every run.

### Statistical Analyses

Data analysis was conducted utilizing SAS software version 8.2 or later (Cary, N.C., USA).

Not all of the possible pairwise comparisons among the 5 feeding groups were of equal interest. Comparisons of primary interest included those between the group receiving Control formula and each of the three experimental formula groups (sn-2 only, sn-2+3 g/L OF and sn-2+5 g/L OF), as well as comparisons between the HM-fed infants and each of the four groups of formula-fed infants. Data on faecal bacteria groups (from FISH method) among formula-fed infants were analysed using analysis of covariance (ANCOVA) with baseline values and type of delivery (vaginal or Caesarean section) included in the model, followed by comparisons of the adjusted mean changes from baseline to week 8 obtained from the ANCOVA. Using a similar ANCOVA model described above, separate analyses were performed at week 8, comparing each formula group to the HM-fed infants. Each of these analyses produced an adjusted mean for the formula group and the HM group, hence a total of 4 different HM adjusted means were calculated. The adjusted means for the HM-fed group are slightly different, depending on which formula-fed group was involved in the pairwise comparison, and the P value for a comparison to the HM-fed group represents the difference between the adjusted mean value for the indicated formula-fed group and the HM-fed adjusted mean value appropriate to that comparison. The analyses of faecal bacterial groups were performed on log transformed values.

Data on counts of faecal bacterial pathogens (from the qPCR method) were analysed in a manner similar to the data on faecal bacterial groups described above. The analyses of faecal bacterial pathogens were performed on log transformed values as well as on ranks of the log transformed values. In both cases, analysis of covariance was done with the baseline value as a covariate and type of delivery as a factor in the model (Note: for 3 of the 4 pathogens, the majority of reported values were 0. All values of 0 were set to 1 for analysis purposes since the log of 0 cannot be calculated). The results of both analyses were similar; however, the analysis based on ranks was preferred since the data were not normally distributed even after log transformation. The bacterial pathogen data presented therefore represent log transformed pathogen counts with P values based on ranks.

The PP population for faecal microbiota (n 170) consisted of formula-fed infants who received only study formula for the duration of the study, or HM-fed infants who consumed only HM; infants who completed all study visits through week 8; infants who had an available measurement at week 8 for the endpoint being analysed; and infants without any major protocol violations (e.g. infants who met the inclusion and exclusion criteria). Infants were excluded from the PP analysis population if they received medications which could potentially impact study endpoints (listed above), and HM-fed infants and formula-fed infants who had ever been breastfed were also excluded if their mothers had used these medications while the infants were being breastfed. The determination of whether the infant had violated the protocol and was excluded from the PP analysis was made before the study was unblinded. Statistical analyses were performed on both the EA and PP populations. Results for faecal bacteria groups were similar in both populations, with statistical significance and conclusions in agreement for nearly all comparisons and parameters, with 4 exceptions. Results from the PP analysis population are presented here since these findings exclude infants who received prohibited medications which could influence study endpoints, including antibiotics. For the 4 comparisons that differ in the EA and PP populations, both results are presented. As bacterial pathogen analyses were conducted as post-hoc explorations of bacterial group differences which were similar in both PP and EA, and because bacterial species may be sensitive to the influence of antibiotics, these analyses were conducted in the PP population only. All statistical tests were two-sided. The level for statistical significance was 0.05.

### Results

### Study Population

In the original study, three hundred formula-fed infants were randomized to receive Control, sn-2, sn-2+3 g/L OF, or sn-2+5 g/L OF. Seventy-five HM-fed infants were enrolled. The current analysis represents a subset of 170 infants with stool samples at baseline and week 8 who comprised the PP analysis population (30-37 per feeding group for the analyses of faecal bacteria groups as well as faecal pH and organic acids, and 23-27 per feeding group with sufficient sample for the analysis of faecal bacteria pathogens). Overall, infants participating in the various feeding groups were generally similar on a variety of baseline characteristics, although HM-fed infants were slightly younger, more likely born via vaginal delivery, and less likely to be male or first born. Compared to the 375 infants in the full study, infants participating in the subset showed no major differences in baseline characteristics. Overall, the proportion of infants in each feeding group with detectable levels of faecal pathogens at week 8 ranged from 81-100% for Klebsiella pneumoniae, 19-54% for EPEC, 0-15% for Clostridium difficile, and 8-40% for Clostridium perfringens.

The results of the study are illustrated by FIGS. 1, 2, 3, 4, and Table 2. In the figures the counts of various bacterial populations are reported (as change from baseline, i.e. population at day 1 of the study). The population counts are reported for the 4 groups: “control” (not a composition of the invention), “sn-2” (not a composition of the invention), “sn-2+3 g/L OF” (composition of the invention) and “sn-2+5 g/LOF” (composition of the invention). The statistically significant differences are indicated by the P values, when present. In Table 2, the counts of various bacterial populations are reported as week 8 values (i.e., population at the end of the study). The population counts are reported for 4 groups: “sn-2” (not a composition of the invention), “sn-2+3 g/L OF” (composition of the invention), “sn-2+5 g/LOF” (composition of the invention), and HM (infants fed human breast milk which is considered the “gold standard” in infant nutrition, not a composition of the invention).

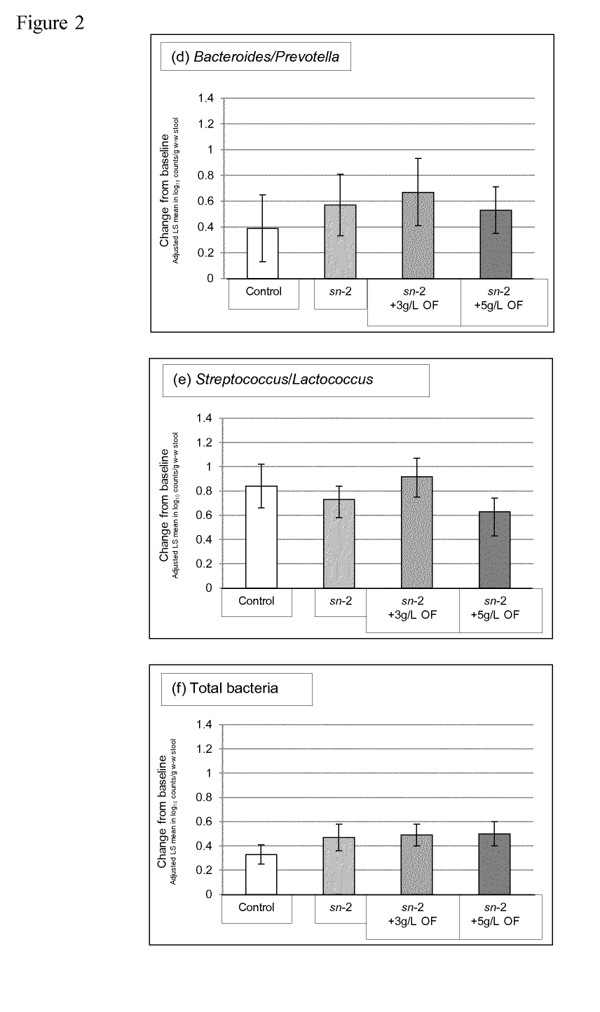

FIGS. 1(a) and 2(f) illustrate that the effect of the invention is specific to potentially pathogenic bacteria groups (especially Enterobacteriaceae and Clostridium—see FIGS. 1 (b) and (c)). The Lactobacillus/Enterococcus counts (FIG. 1(a)) as well as the total bacteria populations (FIG. 2(f)) are not affected by the diets in the 4 groups. Other non-pathogenic groups are not affected by the diets.

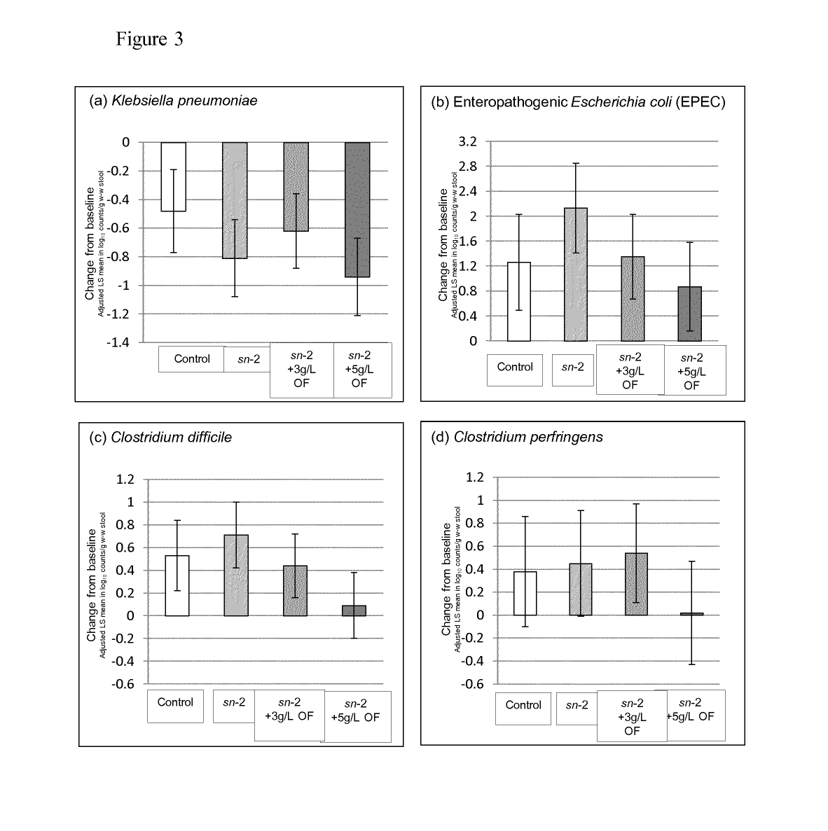

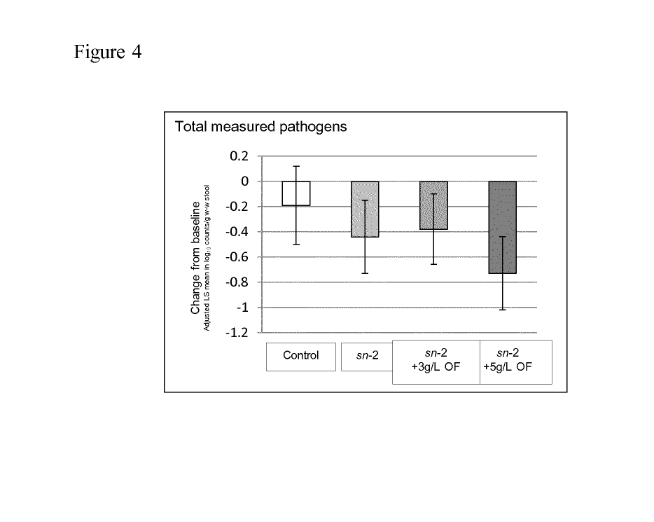

FIGS. 1 and 2 as well as FIGS. 3 and 4 illustrate that
a high sn-2 diet alone does not have any effect in down regulating, decreasing or inhibiting the growth of potentially pathogenic or pathogenic bacterial populations (see for example FIG. 1(c), 3(b), 3(d), columns “sn-2”).
Oligofructose shows an effect at down regulating, decreasing or inhibiting the growth of both potentially pathogenic and pathogenic bacterial populations. That effects is present as a trend at 3 g OF/L (not statistically significant in the conditions of the study) and even more prominent at 5 g OF/L (see in particular FIG. 4, FIG. 3 (b), (c), (d), FIG. 1(c).
The effect is clearly shown on Klebsiella pneumonia, Enteropathogenic Escherichia coli (EPEC), Clostridium difficile and Clostridium perfringens (see FIG. 3) and on total measured pathogens (the sum of the counts of all 4 pathogens) (see FIG. 4). FIG. 1(c) illustrates that the effect is present for the Clostridium taxon, which comprises many different Clostridium species.

TABLE 2
Selected faecal pathogenic bacteria species concentrations^{†} at week 8, by feeding group.

| | sn-2 | | | sn-2 + 3 g/L OF | | | sn-2 + 5 g/L OF | | | HM^{‡} |
|---|---|---|---|---|---|---|---|---|---|---|
| | Value | SE^{§} | p^{\|\|} | Value | SE^{§} | p^{\|\|} | Value | SE^{§} | p^{\|\|} | Values |
| EPEC | 3.70 | 0.56 | <0.001* | 2.71 | 0.56 | 0.024* | 2.26 | 0.54 | 0.16 | 0.85-1.16 |
| Clostridium | 1.59 | 0.36 | 0.034* | 1.33 | 0.32 | 0.17 | 0.73 | 0.30 | 0.85 | 0.36-0.72 |
| perfringens | | | | | | | | | | |
| Total | 5.94 | 0.41 | 0.014* | 5.96 | 0.37 | 0.039* | 5.54 | 0.38 | 0.20 | 4.55-4.73 |
| measured | | | | | | | | | | |
| pathogens^{¶} | | | | | | | | | | |

^{†}Faecal pathogenic bacteria data are expressed as log₁₀ transformed bacteria concentrations (gene copy number counts/g stool wet weight).
^{‡}HM (Human Milk) values shown represent the range of the HM LS Mean values from each pairwise comparison of HM to a formula group.
^{§}Standard errors shown are those for the adjusted means.
^{||}P values are from ANCOVA based on ranks and represent pairwise comparisons to the HM group.
^{¶}Summed counts of all 4 pathogens including Escherichia coli (EPEC), Klebsiella pneumoniae, Clostridium difficile and Clostridium perfringens.
*Results significantly different at P < 0.05 (versus HM group)

Table 2 illustrates that
a high sn-2 diet alone does not have any down-regulating effect on the abundance of pathogenic bacterial populations.
Oligofructose shows an effect on the abundance of pathogenic bacterial populations such that they are more similar to that of HM-fed infants after 8 weeks of feeding. The effect of being similar to HM-fed infants is present at both 3 g OF/L and 5 g OF/L for Clostridium perfringens, and at 5 g OF/L for Enteropathogenic Escherichia coli (EPEC) and total measured pathogens (the sum of the counts of all 4 pathogens). The effect is shown with 3 g OF/L and becomes statistically visible at 5 g OF/L at p<0.05.

Hence, without being bound by the theory, Table 2 shows that the effect of the invention is not only on the down-regulation/inhibition of the pathogenic bacteria, but the invention also promotes an overall abundance of populations that are closer to infants fed with Human Breast Milk (compared to infant fed with conventional formula such as high sn-2 only infant formula).

1. A method for down-regulating, decreasing or inhibiting the growth of pathogenic bacteria in the gut of infants or young children in need of same comprising administering a nutritional composition selected from the group consisting of:
at least 0.4 g or at least 0.7 g Oligofructose/100 kcal of the nutritional composition;
at least 3 g/L or at least 5 g/L of Oligofructose when the composition is a ready-to-drink liquid composition; and
a sufficient amount of oligofructose to obtain respectively at least 3 g/L or 5 g/L of oligofructose in the reconstituted composition when the nutritional composition is a powered or concentrated composition.

2. The method of claim 1 wherein the infants are born at term.

3. The method of claim 1 wherein the infants are 0 to 12 months of age.

4. The method of claim 1 wherein at last 90% of the oligofructose have a degree of polymerisation between 2 and 8.

5. The method of claim 1 wherein the composition comprises triglycerides with high sn-2 palmitate.

6. The method of claim 1 wherein the composition comprises fat and at least 8% of the fat is sn-2 palmitate.

7. The method of claim 1 wherein the composition is an infant formula or a follow-on formula.

8. The method of claim 1 wherein the composition comprises alpha-lactalbumin proteins in an amount of at least 0.3 g/100 kcal or 2.3 g/L of composition.

9. The method of claim 1 wherein the pathogenic bacteria are selected from the group consisting of Klebsiella pneumoniae, E. coli, Enteropathogenic E. coli, Clostridium difficile and/or Clostridium perfringens.

10. The method of claim 1 wherein the down-regulation, decrease or inhibition the growth of pathogenic bacteria is measurable in infants stools.

11. The method of claim 1 wherein the composition is fed or intended to be fed during the first 4 weeks of life.

12. The method of claim 1 wherein the infants have a characteristic selected from the group consisting of born prematurely, born by caesarean section, exhibit unbalanced or abnormal intestinal microbiota and suffer from intestinal infection.

13. The method of claim 1 wherein the down-regulation, decrease or inhibition the growth of pathogenic bacteria in the gut of infants or young children further induces the gut microbiota of the infants or young children to be more similar to the microbiota of exclusively breast-fed infants or young children than microbiota of infants fed from a conventional composition not containing said oligofructose.

14. The method of claim 1 wherein the composition has a characteristic selected from the group consisting of
less than 1 g or less than 2 g Oligofructose/100 kcal of said nutritional composition;
less than 10 g/L or less than 15 g/L of Oligofructose when said composition is a ready-to-drink liquid composition; and
a sufficient amount of oligofructose to obtain respectively less than 10 g/L or less than 15 g/L of oligofructose in the reconstituted composition when the nutritional composition is a powered or concentrated composition.
